(19) United States
(12) Patent Application Publication
(10) Pub. No.: US 2015/0209519 A1
(43) Pub. Date: Jul. 30, 2015

(54) DISPOSABLE INJECTOR FOR INJECTING A LIQUID INTO A PATIENT

(71) Applicant: Injector ApS, Kobenhavn O (DK)

(72) Inventor: Morten Mernøe, Kobenhavn O (DK)

(21) Appl. No.: 14/421,558

(22) PCT Filed: Aug. 14, 2013
(86) PCT No.: PCT/DK2013/050266

§ 371 Date: Feb. 13, 2015

(30) Foreign Application Priority Data
Aug. 14, 2012 (DK) PA 2012 70476

(51) Int. Cl. A61M 5/28; A61M 35/00; A61M 5/00; A61M 5/32; A61M 5/315

(52) U.S. Cl. CPC A61M 5/288; A61M 5/3202; A61M 5/284; A61M 5/282; A61M 5/31596; A61M 5/3294; A61M 5/002; A61M 35/003; A61M 2005/312

(57) ABSTRACT

The present invention relates to a disposable automatic injector for injecting an injection liquid.

### FIELD OF INVENTION

The present invention relates to a disposable automatic injector for injecting an injection liquid.

### BACKGROUND OF INVENTION

Injections are the most common health care procedure worldwide. In developing and transitional countries alone, some 16 thousand million injections are administered each year. More than 90% injections are given for therapeutic purposes while 5 to 10% are given for preventive services, including immunization and family planning.

Unsafe injections place patients at risk of disability and death. Reuse of injection devices without sterilization is of particular concern according to the World Health Organization as it may transmit hepatitis B virus (HBV), hepatitis C virus (HCV) and human immunodeficiency virus (HIV), accounting for 32%, 40% and 5% of new infections in 2000, respectively. In addition, inappropriate and unhygienic use of multi-dose vials may transmit bloodborne pathogens. Moreover, an estimated 4.4% of HIV infections and 39% of HBV and HCV infections are attributed to occupational injury. Among the factors which can cause risks in giving injections or collecting blood are the reuse of injection equipment and the use of non-sterile or reprocessed syringes and needles. (World Health Organization: WHO best practices for injections and related procedures toolkit, 2010).

Accordingly, there is a need for safe injection devices. As the WHO states, “syringes with a reuse prevention feature offer the highest level of safety for injection recipients.” The WHO also recommends using a single-dose vial for each patient, in order to reduce cross-contamination between patients.

In connection with such injectors, there are many different problems related to safety of use, simplicity of use, price, re-utilisation without sterilisation of the injection needle, size as regards logistical considerations in connection with high volume vaccination programs and so on.

A preferred injection device would thus be one that contains a single bolus, is ready to use in order to avoid contamination upon bolus preparation in environments where sterility can be hard to achieve, and cannot be reused. At the same time, shelf-life of the medication to be administered should be as high as possible.

Many different solutions to one or more of these problems have been devised, but there is still a need for an injector that addresses most, if not all, of these problems in a price effective, simple and easy to use way. According to the invention, most, if not all, of these problems are addressed in a novel and effective manner.

### SUMMARY OF INVENTION

The invention relates to a disposable injector comprising:
a housing having an axis extending from a distal end to a proximal end and having a proximal skin contact wall for abutting an injection region, said contact wall being provided with an aperture for receiving an injection needle;
a plunger body having a pressing surface facing in the proximal direction towards said skin contact wall and arranged for moving between a first proximal position and a first distal position, said plunger being axially displaceable between said first distal position, a first intermediate position, a second intermediate position and said first proximal position;
a medicine chamber arranged between said pressing surface and said proximal skin contact wall and axially displaceable together with said surfaces, said medicine chamber being compressible in a non-axial direction by displacing said plunger body towards said needle guide body, and said medicine chamber having a proximal wall portion through which said distal end of said needle can move;
said needle having a sharp distal end received in a passage in said medicine chamber and a sharp proximal end received in said aperture, and being arranged for axial displacement between a third distal position and a third proximal position;
injection displacement means, preferably biasing means, adapted to displace said plunger body from said first distal position to said first proximal position such that said plunger body, said medicine chamber and said needle are displaced in the following sequence:
   said plunger body from said first distal position to said first intermediate position, and said needle from said third distal position to said third proximal position, wherein said proximal end of said needle projects distally outside said skin contact wall, said plunger body from said first intermediate position to said second intermediate position wherein said distal end of said needle has penetrated into said medicine chamber, and
   said plunger body from said second intermediate position to said first proximal position, wherein said medicine chamber has been compressed such that at least a portion of the liquid therein has been pressed out through said proximal end of said needle,
   said needle from said third proximal position to said third distal position.

A disposable automatic injector for injecting an injection liquid and comprising:
a housing having an axis extending from a distal end to a proximal end and having a proximal skin contact wall for abutting an injection region, said contact wall being provided with an aperture for receiving an injection needle having a sharp distal end and a sharp proximal end and arranged axially displaceable in said housing through a passage in said proximal wall;
a container for containing an injection liquid and having a proximal wall portion penetrable by said sharp distal end of said needle,
a medicine chamber containing said container and adapted to be reduced in volume so as to compress said container to press said injection liquid into said needle,
a plunger that can be moved forth and back between a position inside the housing and an end stop outside the housing, whereby a spring positioned between the injection needle and the feeding unit is tensioned for being released when the plunger has been moved forth and back so that the feeding unit is moved forward by the spring force
said injection needle having a sharp distal end received in a passage in said medicine chamber and a sharp proximal end received in said aperture and arranged for axial displacement between a third distal position and a third proximal position, and
a feeding unit, for:
   displacing said medicine chamber and said needle in the proximal direction towards said proximal wall such that said needle is extended outside said proximal wall,
   causing said sharp distal end of said needle to penetrate into said container,
   reducing the volume of said container, and, when said container is completely empty, allowing said chamber to pass said feeding unit in the distal direction
   displacing said needle, said container and said compression chamber in the distal direction away from said proximal wall such that the sharp proximal end of said needle becomes located within said housing,
      characterized in that the container is placed in the medicine chamber between a bottom surface and a top surface of the medicine chamber, wherein said surfaces are hinged in the proximal wall thereby providing a compressible medicine chamber movable along the axis of the injector in response to movement of the displacement means, said medicine chamber compressing the container when moved towards said proximal wall.

In the present context, the term ‘feeding unit’ is synonymous with ‘injection displacement means’.

### DESCRIPTION OF DRAWINGS

FIGS. 1-7 are schematic views of a currently preferred embodiment of an injector according to the invention illustrating sequential positions of the various elements of the injector when carrying out an injection.

FIG. 8 shows a detailed view of part of an embodiment.

### DETAILED DESCRIPTION OF THE INVENTION

### Definitions

### Active Substance

As understood herein, an active substance or pharmaceutically active substance is any substance or compound, such as a molecule, a macromolecule, a protein, an antibody, a vaccine, a nucleic acid molecule, an antigen, a lipid, or any compound which has an activity on the body, its metabolism, or its immune response.

### Breakable

The term “breakable” as used herein in reference to the peel section separating the two chambers of the container is to be understood as follows. Said peel section is such that exertion of an external force on the medicine chamber in which the container is placed results in the peel section being broken, ruptured, torn or punctured. Rupturing or breaking of the peel section allows communication of flow between the two chambers of the container. The peel section is not restorable once broken, i.e. breaking or rupturing of the peel section is not reversible.

### Chamber

A chamber as understood herein is a compartment which is closed and can contain a liquid or solid substance.

### Distal

The term distal refers herein to the physical location of a part of the injector. A part of the injector is located distally when it is in the half of the injector opposite to the skin contacting surface.

### Impermeable/Water-Tight

The separation wall of the container described herein is impermeable or water-tight, i.e. it does not allow communication or exchange of the substances contained in the two chambers surrounding said separation wall, unless it is broken.

### Needle

A needle as understood herein refers to a needle for injecting a liquid composition, such as a pharmaceutical composition. Such a needle may have zero, one or two sharp ends In the case of needles with two sharp ends, the sharp distal end can be used to rupture a membrane which is comprised in the means for attaching a needle, for example on the wall of the second chamber, so that the composition contained in the container can flow within the needle. The needle can be capped at the proximal end, to avoid accidental sticking or spillage of the liquid composition of the container. The means for attaching a needle comprise conventional means known in the art: the needle may be glued to the container, encased in the container, or the means for attaching a needle is such as a luer lock.

### Pressure

Pressure is the effect of a force applied to a surface. Pressure is the amount of force acting per unit area. The symbol of pressure is p. The internal pressures herein referred to relate to the pressures within the chambers of the container. The chambers may be filled and/or sealed under conditions where the pressure is lower than the atmospheric pressure, so that their internal pressure is inferior to 1 atm (or 1.0197×10⁻⁵ Pa).

### Proximal

The term proximal refers herein to the physical location of a part of the injector. A part of the injector is located distally when it is in the same half of the injector as the skin contacting surface.

### Retraction

The term retraction is herein used in reference to a movement of the plunger from a proximal position where it is located within the housing of the injector to a distal position, where it is located outside the housing of the injector. The plunger is retracted when the user exerts a pulling force on it.

### DETAILED DESCRIPTION OF THE INVENTION

The invention relates to a disposable injector comprising:
a housing having an axis extending from a distal end to a proximal end and having a proximal skin contact wall for abutting an injection region, said contact wall being provided with an aperture for receiving an injection needle;
a plunger body having a pressing surface facing in the proximal direction towards said skin contact wall and arranged for moving between a first proximal position and a first distal position, said plunger being axially displaceable between said first distal position, a first intermediate position, a second intermediate position and said first proximal position;
a medicine chamber arranged between said pressing surface and said proximal skin contact wall and axially displaceable together with said surfaces, said medicine chamber being compressible in a non-axial direction by displacing said plunger body towards said needle guide body, and said medicine chamber having a proximal wall portion through which said distal end of said needle can move;
said needle having a sharp distal end received in a passage in said medicine chamber and a sharp proximal end received in said aperture, and being arranged for axial displacement between a third distal position and a third proximal position;
injection displacement means, preferably biasing means, adapted to displace said plunger body from said first distal position to said first proximal position such that said plunger body, said medicine chamber and said needle are displaced in the following sequence:
   said plunger body from said first distal position to said first intermediate position, and said needle from said third distal position to said third proximal position, wherein said proximal end of said needle projects distally outside said skin contact wall, said plunger body from said first intermediate position to said second intermediate position wherein said distal end of said needle has penetrated into said medicine chamber, and
   said plunger body from said second intermediate position to said first proximal position, wherein said medicine chamber has been compressed such that at least a portion of the liquid therein has been pressed out through said proximal end of said needle,
   said needle from said third proximal position to said third distal position.

A disposable automatic injector for injecting an injection liquid and comprising:
a housing having an axis extending from a distal end to a proximal end and having a proximal skin contact wall for abutting an injection region, said contact wall being provided with an aperture for receiving an injection needle having a sharp distal end and a sharp proximal end and arranged axially displaceable in said housing through a passage in said proximal wall;
a container for containing an injection liquid and having a proximal wall portion penetrable by said sharp distal end of said needle,
a medicine chamber containing said container and adapted to be reduced in volume so as to compress said container to press said injection liquid into said needle,
a plunger that can be moved forth and back between a position inside the housing and an end stop outside the housing, whereby a spring positioned between the injection needle and the feeding unit is tensioned for being released when the plunger has been moved forth and back so that the feeding unit is moved forward by the spring force
said injection needle having a sharp distal end received in a passage in said medicine chamber and a sharp proximal end received in said aperture and arranged for axial displacement between a third distal position and a third proximal position, and
a feeding unit, for:
   displacing said medicine chamber and said needle in the proximal direction towards said proximal wall such that said needle is extended outside said proximal wall,
   causing said sharp distal end of said needle to penetrate into said container,
   reducing the volume of said container, and, when said container is completely empty, allowing said chamber to pass said feeding unit in the distal direction
   displacing said needle, said container and said compression chamber in the distal direction away from said proximal wall such that the sharp proximal end of said needle becomes located within said housing,
      characterized in that the container is placed in the medicine chamber between a bottom surface and a top surface of the medicine chamber, wherein said surfaces are hinged in the proximal wall thereby providing a compressible medicine chamber movable along the axis of the injector in response to movement of the displacement means, said medicine chamber compressing the container when moved towards said proximal wall.

In the present context, the term ‘feeding unit’ is synonymous with ‘injection displacement means’.

The sequence through which said plunger body, said medicine chamber and said needle are displaced may be performed automatically through the action of the displacement means.

In some embodiments the injector further comprises a needle guide body provided with a passage for receiving and guiding said injection needle.

In a preferred embodiment the needle guide body has an abutment surface facing in a distal direction towards said pressing surface and arranged for axial displacement between a second distal position, a third intermediate position and a second proximal position.

In a further preferred embodiment said injection displacement means, preferably biasing means, is adapted to displace said plunger body from said first distal position to said first proximal position such that said plunger body, said medicine chamber, said guide body and said needle are displaced in the following sequence:
said plunger body and said guide body from said first distal position and said second distal position, respectively, to said first intermediate position and said third intermediate position, respectively, and said needle from said third distal position to said third proximal position, wherein said proximal end of said needle projects distally outside said skin contact wall, said plunger body and said guide body from said first intermediate position and said third intermediate position, respectively, to said second intermediate position and said second proximal position, respectively, wherein said distal end of said needle has penetrated into said medicine chamber,
said plunger body from said second intermediate position to said first proximal position, wherein said medicine chamber has been compressed such that at least a portion of the liquid therein has been pressed out through said proximal end of said needle, and
said guide body and said needle from said third intermediate position and said third proximal position, respectively, to said second distal position and said third distal position, respectively.

The sequence through which said plunger body, said medicine chamber, said guide body and said needle are displaced may be performed automatically through the action of the displacement means.

It is preferred that the medicine chamber:
further contains a container for containing an injection liquid and having a proximal wall portion penetrable by said sharp distal end of said needle;
is adapted to be reduced in volume so as to compress said container to press said injection liquid into said needle.

In some embodiments, the injection liquid is a pharmaceutically active liquid.

Having a separate container inside the medicine chamber allows separate manufacturing of the container and of the remaining parts of the injector, thus conveniently allowing containers with various contents to be used with the same injector. As a result a variety of injectors may be produced containing different injection liquids.

The invention further relates to a disposable automatic injector for injecting an injection liquid and comprising:
a cylindrical housing with a distal wall and a proximal wall having a proximal surface for abutting the surface of an injection site,
an injection needle having a sharp distal end and a sharp proximal end and arranged axially displaceable in said housing through a passage in said proximal wall
a compressible container for containing an injection liquid and having a proximal wall portion penetrable by said sharp distal end of said needle,
a compression chamber containing said container and adapted to be reduced in volume so as to compress said container to press said injection liquid into said needle, and
displacement means, preferably biasing means, for:
   displacing said compression chamber and said needle in the proximal direction towards said proximal wall such that said needle is extended outside said proximal wall,
   causing said sharp distal end of said needle to penetrate into said container,
   reducing the volume of said compression chamber, and
   displacing said needle, said container and said compression chamber in the distal direction away from said proximal wall such that the sharp proximal end of said needle becomes located within said housing.

In some embodiments, the needle guide body is immobile within the housing. In other embodiments, the needle guide body is arranged so that movement of the needle guide body, for example by application of a pressure on its distal abutment surface, results in movement of the needle in the same direction. For example the needle guide body may be connected to the needle.

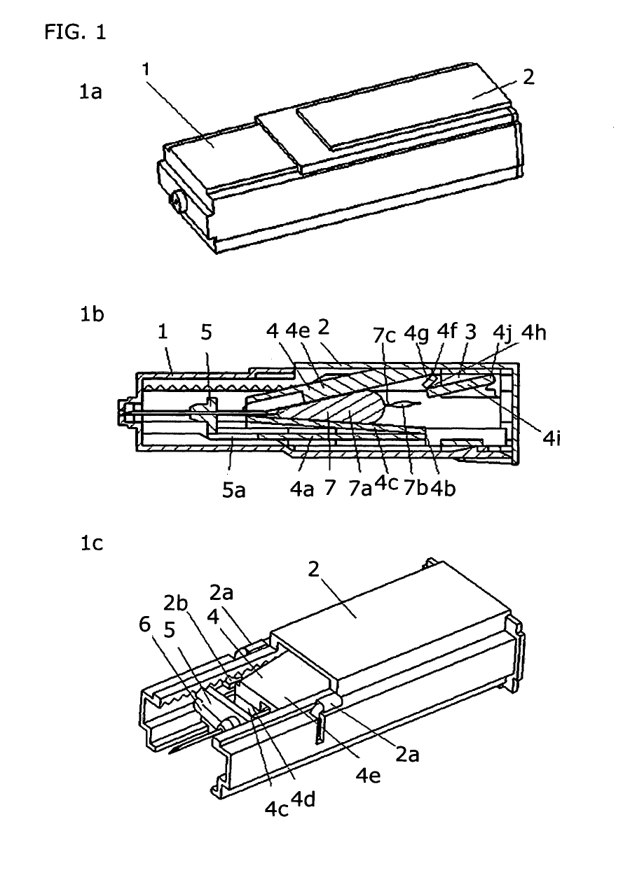

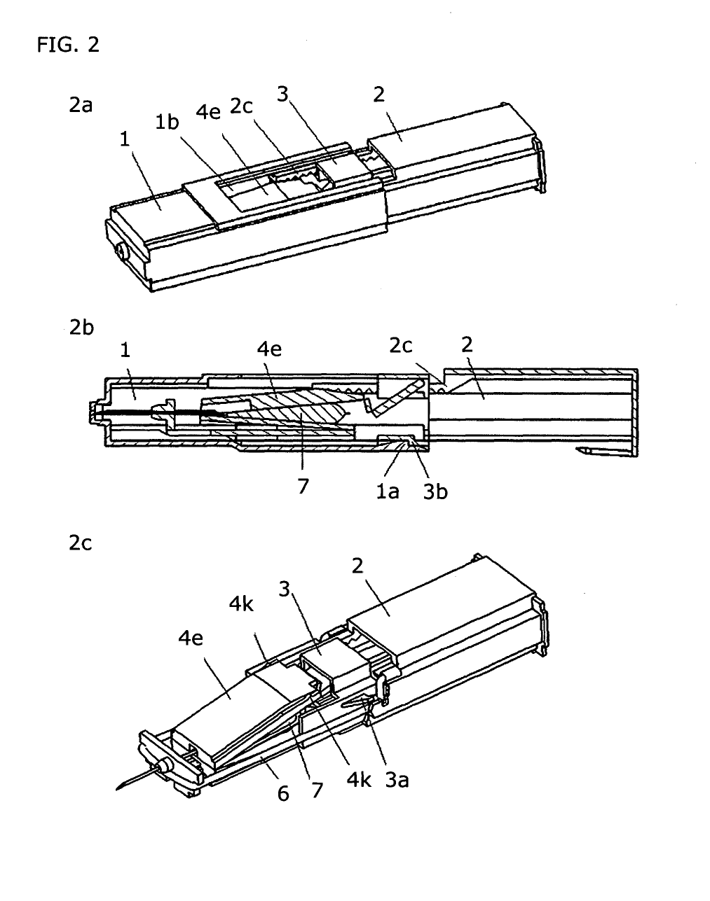

The medicine chamber may comprise a top and a bottom surface (4a and 4c, respectively, FIG. 1b). At least one of the top and bottom surfaces of the medicine chamber may be connected to the plunger body. In one embodiment, application of a force through the injection displacement means results in a pressure being exerted on at least one of the top and bottom surface of the medicine chamber, thus resulting in compression of the medicine chamber in a non-axial manner. In some embodiments, only one surface is connected to the plunger and the other surface is immobile relative to the housing. In some embodiments, connection of the at least one of the top and bottom surface of the medicine chamber is achieved by at least one hinge. In some embodiments, at least one of the surfaces of the medicine chamber is hinged to the housing so that movement of the plunger from a proximal position to a distal position results in the hinged surface exerting a pressure on the container placed within the medicine chamber (FIG. 2). The top and the bottom surfaces may both be hinged to the housing to allow the container to be compressed from two directions at once. Other means of attaching the at least one of the surfaces of the medicine chamber are means known in the art, such as a spring, of which one end is attached to the plunger and the other to the at least one surface.

The top and bottom surfaces of the medicine chamber may be planar or curved. Preferably, said surfaces are not parallel and their axes intersect at the proximal end of the injector. The top and bottom surfaces should be so arranged relative to each other that they may at least partly overlap, i.e. they should be so arranged that exertion of a pressure on the walls of the housing enables compression of the medicine chamber by the top and bottom surfaces.

In some embodiments, a spring is positioned between the injection needle and the displacement means. When the plunger is retracted, for example when the user pulls the plunger, the spring is tensioned for being released so that the displacement means is moved forward by the spring force. In some embodiments, said spring is made of rubber. In other embodiments, the displacements means comprises a rubber band.

Also disclosed are embodiments in which the container is made of a plastic film. Preferably, the region of the container which is to be penetrated by the sharp distal end of the needle is provided with a sealing film. Upon penetration of the container by the distal end of the needle, the sealing film seals against the side surface of the needle, thus ensuring that no injection liquid is spilled on the sides of the needle. Such sealing film may for example be made of rubber or of polymers as is known in the art.

In some embodiments, the injection liquid is a pharmaceutically active liquid, such as a vaccine.

It is another object of the invention to provide a disposable injector wherein said container for containing an injection liquid consists of:
a first chamber containing a dry matter;
a second chamber containing a liquid;
a peel section separating the first and the second chamber;
   wherein the peel section is breakable.

In preferred embodiments, the first and second chambers are impermeable, so that the dry matter and the liquid they contain may not spill in the medicine chamber in which the container is placed. The peel section is preferably also impermeable, thus contact between the liquid and the dry matter is prevented as long as the plunger is not being retracted. The peel section is breakable, so that upon retraction of the plunger, breakage of the peel section allows contacting of the dry matter of the first chamber with the liquid of the second chamber, resulting in a liquid composition or injection liquid.

In some embodiments, the dry matter comprises an active substance, such as a pharmaceutically active substance, such as a vaccine. The liquid contained in the second chamber may comprise an active substance or a solvent. In some embodiments, the liquid is a solvent, such that it results in an active solution upon mixing with the active substance of the first chamber.

In some embodiments, complete retraction of the plunger results in mixing of the dry matter and the liquid. For example, the plunger may be provided with protuberances or teeth, so that upon retraction the protuberances get in contact with the top of the medicine chamber, thus resulting in vibrations facilitating mixing of the dry matter and the liquid.

Pharmaceutically active substances have a longer storage life when in a dry form. Active substances in dry form also often display higher stability when confronted to uncontrolled environmental conditions such as high or low temperature, temperature variations or pressure variations. Thus embodiments with two separate chambers may be particularly relevant when the injection liquid is a pharmaceutically active substance, as it may extend the shelf life of the injector.

In some embodiments, the proximal skin contact wall of the housing is provided with an antiseptic means intended for being applied to the injection site. Such antiseptic means may be any means known in the art such as commercial antiseptics, for example trichlorophenylmethyliodosalicyl. In embodiments where the antiseptic means is volatile, the skin contact wall may be covered by an air-tight membrane or peel section to prevent its evaporation.

The invention also relates to an injector, wherein a cross-section of the housing is of rectangular shape. The cross-section of the housing may thus be square, rectangular, and have sharp or round edges. Such embodiments present several advantages compared to injectors where a cross-section of the housing is rectangular. The dead volume of the injector, i.e. the volume which is not essential for its proper functioning, is reduced, thus reducing the total volume of the injector as compared to a circular injector containing the same volume of medicine, and thus allowing storage in a lesser space. The rectangular shape allows the user to get a better grip on the housing as it provides flat, lateral surfaces. The housing may also be equipped with grips on its lateral faces in order to further facilitate manipulation.

In some embodiments, the housing is provided with a window, which may allow visualisation of the medicine chamber, in order to allow the user to check for integrity of the medicine bag, or for correct mixing of the contents of the medicine chamber.

### EXAMPLES

In this section some preferred embodiments are described in details, solely by way of example. The shown embodiments shall not be construed as limiting for the scope of the invention.

### Example 1

This preferred embodiment will be described in detail in the below and is shown in FIGS. 1 to 8, where:
Housing 1
Housing lock 1a
Housing hole 1b
Curved path in housing 1c
Needle stop inside housing 1d
Housing feeding stop 1e
Plunger 2
Spring attachments 2a
Plunger hook 2b
Plunger teeth 2c
Plunger trigger 2d
Displacement means 3
Displacement means hooks 3a
Displacement means recess 3b
Medicine chamber 4
Medicine chamber pal 4a
Medicine chamber bottom 4c
Medicine chamber top 4e
Medicine chamber intermediate section 4g
Medicine chamber connecting rod 4i
Needle 5
Needle plastic part 5a
Needle rear end 5b
Spring or rubber band 6
Medicine bag 7
Medicine bag liquid chamber 7a
Medicine bag dry chamber 7b
Medicine bag peel section 7c

Referring to FIGS. 1a, 1b and 1c there is shown the syringe in its original position. FIG. 1a shows the syringe from the outside in perspective. FIG. 1b shows the syringe in section through the centre line. FIG. 1c shows the syringe in part, separated. The syringe comprises a housing 1, a plunger 2, a feed unit 3, a medicine chamber 4, a needle 5, a spring or rubber band 6, and a medicine bag 7. The spring or rubber band 6 is shown in FIG. 1a-c inactivated and secured on the plunger 2 in the attachment points 1a.

From there, the spring or rubber band 6 is passed around hooks 2b (only one is visible in FIG. 1c) on the plunger 2 and the needle 5. The needle 5 is thus secured. Needle 5 has a plastic portion 5a abutting the medicine chamber pal 4a whereby it is prevented from sliding backwards. Medicine chamber pal 4a is hinged to the 5 medicine chamber bottom 4c. The bottom of the medicine chamber 4c can move in the axial direction. In the bottom of the medicine chamber 4c is a medicine bag 7 attached so that it is caught between medicine chamber bottom 4c and medicine chamber top 4e which is hinged at point 4d. Medicine bag 7 consists of two chambers: the liquid chamber 7a and dry chamber 7b separated by a peel section 7c. Medicine chamber top 4e is in FIG. 1a-c in a fully open position but controlled by the plunger 2 and hinged at the point 4f to the medicine chamber via the intermediate section 4g, which is hinged at the point 4h to the medicine chamber connecting rod 4i, which in turn contacts the medicine chamber 4 in the point 4j.

FIGS. 2a-c show the syringe with the plunger fully retracted.

FIGS. 2a show the syringe from the outside in perspective. FIG. 2b shows the syringe in section through the centre line. FIG. 2c shows the syringe in part, separated.

Upon retraction the plunger teeth 2c hits the top of the medicine chamber 4e in points 4k, whereby the medicine chamber top 4e is pressed down towards the medicine bag 7 so that the peel section 7c (see FIG. 1b) is broken and the liquid from the chamber 7a is mixed with dry matter 7b. By further retraction of the plunger 2 the plunger teeth 2c slide along the medicine chamber top 4e in points 4k, whereby the contents of the medicine bag 7 are vibrated for better mixing of liquids and solids. In the fully retracted position the spring or rubber band 6 attaches to the hooks 3a of the displacement means 3 (only one of the two are visible in FIG. 2c). Since the displacement means 3 is locked when the recess 3b rests against the housing lock 1a the plunger 2 remains in the fully open position. It is possible to inspect the medicine bag 7 through the hole 1b in the housing 1. The syringe is now ready for injection, and is placed against the skin.

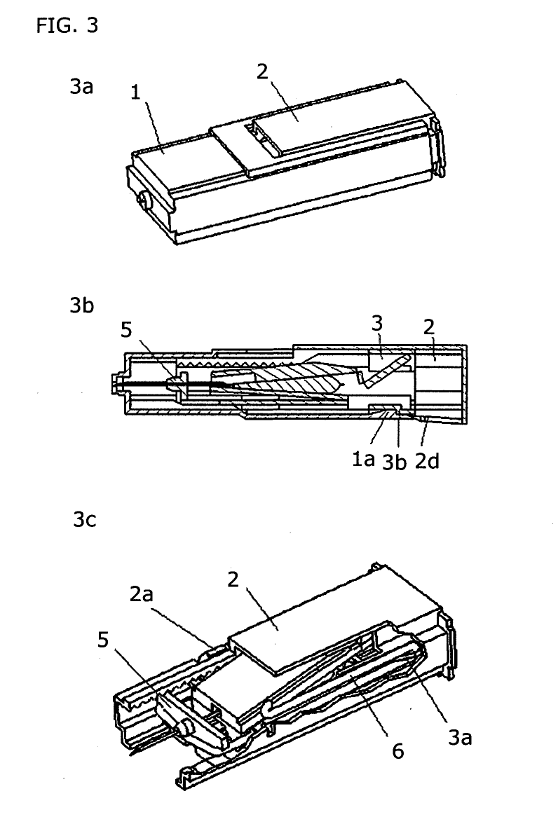

FIGS. 3a-c show the syringe with the plunger 2 pushed so that the feeding device recess 3b is almost released from the housing lock 1 by the plunger 2 triggering 2d squeezed in between them. FIG. 3a shows the syringe from the outside in perspective. FIG. 3b shows the syringe in a section through the centre line. Also in FIG. 3c the syringe partially separated. The spring or rubber band 6 is now fully activated and its course is now from the attachment points 2a to the feed device hooks 3a and towards the needle 5.

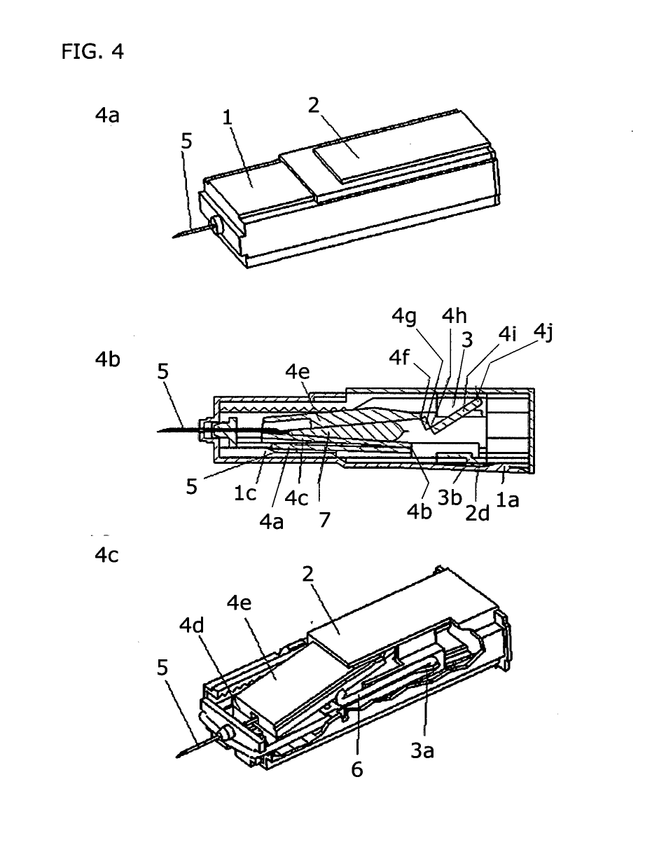

FIGS. 4a-c shows the syringe with the needle released into the skin.

FIG. 4a shows the syringe from the outside in perspective. FIG. 4b shows the syringe in section through the centre line. FIG. 4c shows the syringe in part, separated. The housing lock 1a has been released from the recess 3b by inserting the plunger trigger 2d there between. The spring or rubber band 6 pulls the displacement means 3 forward, whereby it pushes the connecting rod 4i of medicine chamber via the hinge 4j. The connecting rod 4i pushes in a forward and downward direction on the intermediate section 4g of the medicine chamber via hinge 4h. The intermediate section 4g of the medicine chamber pulls the medicine chamber top 4e in a forward and downward right direction via the hinge 4f. Medicine chamber top 4e is prevented by medicine bag 7 from moving down and hence it moves forward and pulls the medicine chamber bottom through the hinge 4d. Medicine chamber bottom 4c pushes the medicine chamber pal 4a forward via the hinge 4b. Medicine chamber pal 4a pushes the needle 5 forward to the medicine chamber pal 4a and hits the curve path 1c of the housing, whereby the medicine chamber pal 4a releases its engagement in the needle plastic portion 5a.

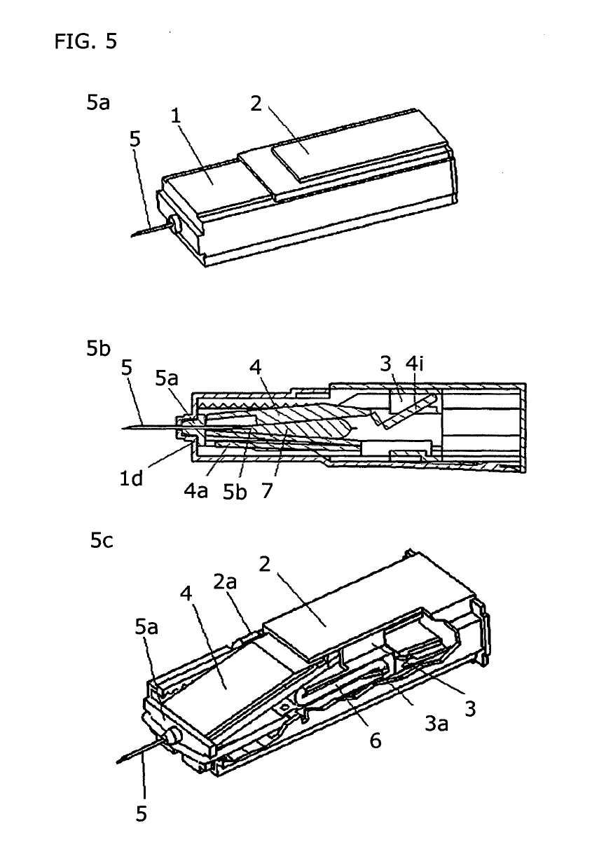

FIGS. 5a-c show the syringe with the medicine chamber completely released and ready to inject the medicine. FIG. 5a shows the syringe from the outside in perspective. FIG. 5b shows the syringe in section through the centre line, and in FIG. 5c the syringe is partially separated.

The displacement means 3 continuously pushes forward on the connecting rod 4i of the medicine chamber due to the spring force of the spring or rubber band 6 that acts between the feed device hooks 3a and the spring attachments 2a. Thereby the medicine chamber 4 continues forward and when the medicine chamber pal 4a no longer pushes the needle 5 onwards, the medicine bag frame 7 is punctured by the needle rear end 5b. The needle continues until stopped by the needle stop 1d inside the housing, while the medicine chamber continues until it hits the needle plastic part 5a.

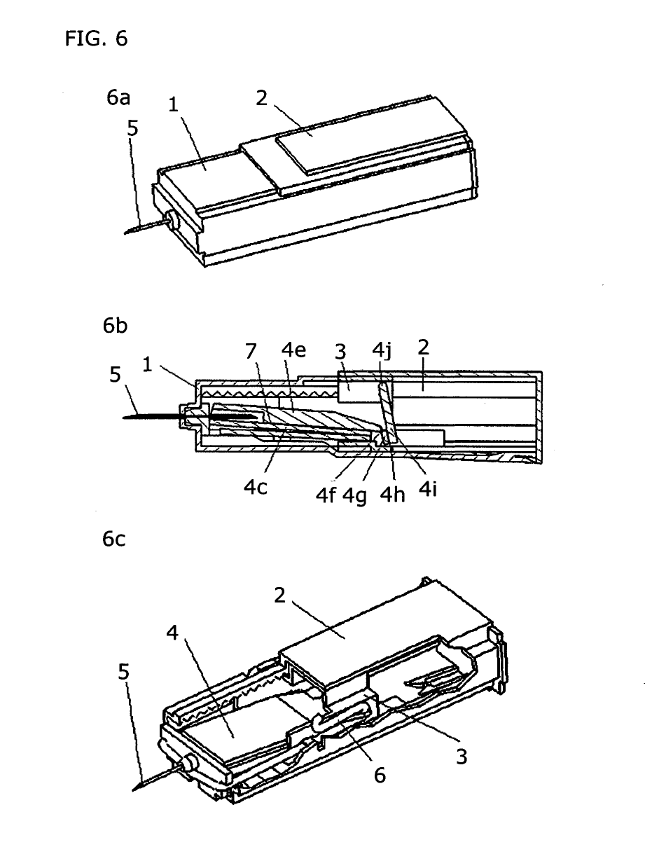

FIGS. 6a-c show the syringe when the medicine bag 7 has been emptied and the connecting rod 4i of the medicine chamber has passed. FIG. 6a shows the syringe from the outside in perspective. FIG. 6b shows the syringe in section through the center line. FIG. 6c shows the syringe partially separated.

Since the displacement means 3 continuously exerts a forward directed force from the bearing 4j on the medicine chamber connecting rod 4i, the connecting rod transforms this force to both a forward and downward force on the medicine chamber top 4e via the intermediate section 4g and the hinges 4f and 4h. As the compression of the chamber 4 is prevented by the needle 5 from moving further 25 forward the chamber top 4e is pressed down towards the medicine bag 7. The length of the medicine chamber connecting rod is adapted in such a way that when the medicine chamber connecting rod reaches the vertical position, the medicine bag 7 fully compressed, and thus emptied.

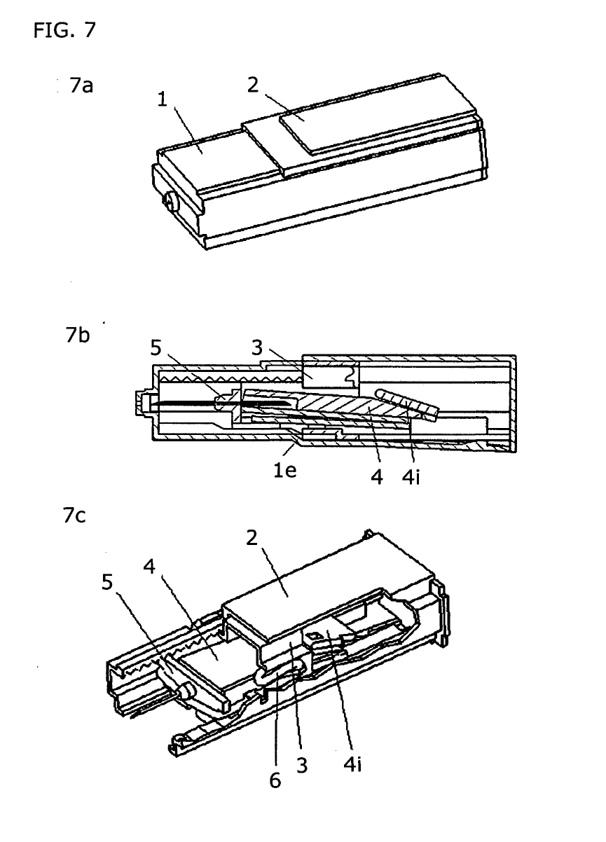

FIGS. 7a-c show the syringe in its end position.

FIG. 7a shows the syringe from the outside in perspective. FIG. 7b shows the syringe in section through the centre line. FIG. 7c shows the syringe partially separated. When medicine bag 7 is emptied the rotation of the connecting rod 4i relative to the vertical position enables the connecting rod 4i, and thus the entire medicine chamber 4, to move backward through the displacement means. The spring force of the spring or rubber band 6 that acts between the needle 5 and the displacement means pulls the needle rearward into the housing. Since the spring is longer than in the starting position it will pull the needle farther than in the original position, thus avoiding contamination risks from used syringes. The injection is now complete and the syringe can be disposed. The syringe cannot be reused since the connecting rod is no longer in engagement with the feeding device.

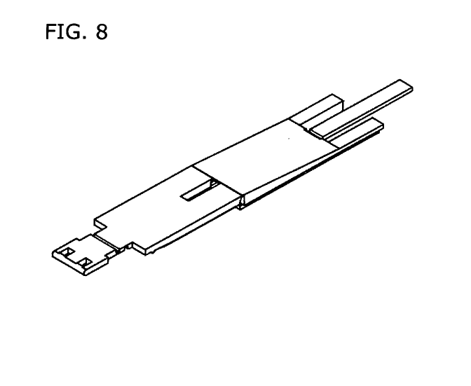

FIG. 8 shows an embodiment of the medicine chamber 4 in an open position, before assembly within the injector. In this embodiment the whole medicine chamber is manufactured from one piece.

1. A disposable injector comprising:
a housing having an axis extending from a distal end to a proximal end and having a proximal skin contact wall for abutting an injection region, said contact wall being provided with an aperture for receiving an injection needle;
a plunger body having a pressing surface facing in the proximal direction towards said skin contact wall and arranged for moving between a first proximal position and a first distal position, said plunger being axially displaceable within said housing between said first distal position, a first intermediate position, a second intermediate position and said first proximal position;
a medicine chamber arranged between said pressing surface and said proximal skin contact wall and axially displaceable together with said surfaces, said medicine chamber being compressible in a non-axial direction by displacing said plunger body towards said skin contact wall, and said medicine chamber having a proximal wall portion through which said distal end of said needle can move;
said needle having a sharp distal end received in a passage in said medicine chamber and a sharp proximal end received in said aperture, and being arranged for axial displacement between a third distal position and a third proximal position;
injection displacement means, preferably biasing means, adapted to displace said plunger body from said first intermediate position to said first proximal position;
wherein said plunger body, said medicine chamber and said needle are displaced in the following sequence:
said plunger body from said first distal position to said first intermediate position, and said needle from said third distal position to said third proximal position, wherein said proximal end of said needle projects distally outside said skin contact wall,
said plunger body from said first intermediate position to said second intermediate position wherein said distal end of said needle has penetrated into said medicine chamber,
said plunger body from said second intermediate position to said first proximal position, wherein said medicine chamber has been compressed such that at least a portion of the liquid therein has been pressed out through said proximal end of said needle, and
said needle from said third proximal position to said third distal position.

2. The injector according to claim 1, further comprising a needle guide body provided with a passage for receiving and guiding said injection needle.

3. The injector according to claim 1, wherein the needle guide body has an abutment surface facing in a distal direction towards said pressing surface and arranged for axial displacement between a second distal position, a third intermediate position and a second proximal position.

4. The injector according to claim 3, wherein said injection displacement means, preferably biasing means, is adapted to displace said plunger body in said proximal direction such that said plunger body, said medicine chamber, said guide body and said needle are displaced in the following sequence:
said plunger body and said guide body from said first distal position and said second distal position, respectively, to said first intermediate position and said third intermediate position, respectively, and said needle from said third distal position to said third proximal position, wherein said proximal end of said needle projects distally outside said skin contact wall, said plunger body and said guide body from said first intermediate position and said third intermediate position, respectively, to said second intermediate position and said second proximal position, respectively, wherein said distal end of said needle has penetrated into said medicine chamber,
said plunger body from said second intermediate position to said first proximal position, wherein said medicine chamber has been compressed such that at least a portion of the liquid therein has been pressed out through said proximal end of said needle, and
said guide body and said needle from said third intermediate position and said third proximal position, respectively, to said second distal position and said third distal position, respectively.

5. The disposable injector according to claim 1, wherein the needle guide body is arranged so that movement of said needle guide body results in movement of the needle in the same direction.

6. The injector according to claim 1, wherein the medicine chamber:
further contains a container for containing an injection liquid and having a proximal wall portion penetrable by said sharp distal end of said needle;
is adapted to be reduced in volume so as to compress said container to press said injection liquid into said needle.

7. The injector according to claim 6, wherein:
said plunger is capable of activating said displacement means;
said container is placed in said medicine chamber between a bottom surface and a top surface of the medicine chamber, wherein said surfaces are arranged so that a pressure can be exerted on them in a direction which is not axial, thereby providing a compressible medicine chamber movable along the axis of the injector in response to movement of the displacement means, said medicine chamber compressing the container when moved towards said proximal wall.

8. The disposable injector according to claim 7, wherein at least one of the bottom and top surfaces of the medicine chamber is connected to the plunger so that movement of the plunger results in the plane comprising said at least one surface to rotate in a non-axial direction.

9-11. (canceled)

12. The disposable injector according to claim 8, wherein connection of the at least one surface to the plunger is achieved by the means of at least one hinge.

13. The disposable injector according to claim 7, whereby a spring positioned between the injection needle and the displacement means is tensioned for being released when the plunger is retracted so that the displacement means is moved forward by the spring force.

14. The disposable injector according to claim 1, wherein the injection displacement means comprises a rubber band.

15. (canceled)

16. The disposable injector according to claim 1, wherein said container is a bag made of a plastic film.

17. The disposable injector according to claim 1, wherein the region of the container, which is to be penetrated by said sharp distal needle end is provided with a sealing film, for instance made of rubber, which is intended to seal against the side surface of said needle, when said needle has penetrated into said bag.

18. The disposable injector according to claim 1, wherein said container consists of:
a first chamber containing a dry matter;
a second chamber containing a liquid;
a peel section separating the first and the second chamber;
wherein the peel section is breakable.

19. The injector according to claim 14, wherein breakage of the peel section occurs upon retraction of the plunger, whereby the dry matter contained in the first chamber and the liquid contained in the second chamber are contacted, thus resulting in a liquid composition.

20. The injector according to claim 14, wherein the dry matter comprises an active substance.

21-22. (canceled)

23. The injector according to claim 14, wherein the liquid contained in the second chamber comprises an active compound or a solvent.

24. The injector according to claim 17, wherein the liquid contained in the second chamber is a solvent.

25. (canceled)

26. The injector according to claim 14, wherein further retraction of the plunger results in vibrations allowing for mixing of the dry matter and of the liquid.

27. The injector according to claim 1, wherein the proximal skin contact wall of the housing is provided with an antiseptic means intended for being applied to the injection site.

28-30. (canceled)
